(19) United States
(12) Patent Application Publication
(10) Pub. No.: US 2010/0221803 A1
(43) Pub. Date: Sep. 2, 2010

(54) MATERIAL CONVERSION METHOD USING CELLULOSE-BASED BIOMASS

(75) Inventors: Takafumi Shimoda, Niigata (JP); Kozo Nishibori, Niigata (JP); Yosuke Baba, Niigata (JP)

Correspondence Address: LUCAS & MERCANTI, LLP, 475 PARK AVENUE SOUTH, 15TH FLOOR, NEW YORK, NY, 10016, US

(73) Assignee: YUKIGUNI MAITAKE CO., LTD., Minamiuonuma-shi, Niigata (JP)

(21) Appl. No.: 12/681,999

(22) PCT Filed: Oct. 6, 2008
(86) PCT No.: PCT/JP2008/068168

§ 371 Date: Apr. 7, 2010

(30) Foreign Application Priority Data
Oct. 10, 2007 (JP) 2007-264041

(51) Int. Cl. C12P 7/06; C12P 1/00

(52) U.S. Cl. 435/161; 435/41

(57) ABSTRACT

It is an object of the present invention to develop a conversion method for easily obtaining a useful substance such as ethanol from a cellulose-based biomass at a good yield via enzyme reaction and microbial fermentation. It has been found that the conversion efficiency can be increased with the use of hard balls or the like when a cellulose-based biomass is converted into sugar with the use of enzyme(s) and then a useful substance such as ethanol with the use of microorganism(s).

### TECHNICAL FIELD

The present invention relates to a conversion method for easily obtaining a useful substance such as ethanol from a cellulose-based biomass with good efficiency via an enzyme reaction and microbial fermentation.

### BACKGROUND ART

At present, it is said that global reduction of carbon dioxide is necessary for prevention of global warming. Under such circumstances, the use of energy derived from unused biomass, and particularly a cellulose-based biomass, has been gaining attention. One reason for that is that such biomass can be so-called carbon-neutral biomass. Specifically, biomass contains carbon originally derived from atmospheric carbon dioxide that has been absorbed or fixed by plants. Thus, offsetting of carbon dioxide emissions (±0) resulting from energy extraction from such biomass is achieved by regenerating plants that can absorb emitted carbon dioxide. In addition, since it is possible to obtain fuel material such as ethanol or methane gas from biomass, biomass has been expected to replace fossil fuels that will be depleted in the future (Non-Patent Document 1).

At present, biomass-derived ethanol (bioethanol) has been actively produced in Brazil with the use of sugar from sugarcane and in the U.S. with the use of edible parts of maize. It has been used in practice as a gasoline alternative in each country. Such forms of bioethanol have been produced from portions that can also be used for food for humans or feed for livestock. Therefore, if bioethanol is increasingly used as a fuel material, there is a concern that food prices might sharply rise, for example.

Therefore, a cellulose-based biomass produced from non-edible plants or woods has been gaining attention as a bioethanol material. However, it is difficult to convert cellulose-based biomass into fuel material. This is because that cellulose used as a starting material for a fuel substance has high crystallinity, and that cellulose contained in such biomass, from which fuel material is obtained, is surrounded by persistent lignin, and thus it is difficult to use cellulose. Therefore, for the use of cellulose in a cellulose-based biomass, it is necessary to, for example, reduce the crystallinity and remove lignin and the like from cellulose contained in such biomass so as to obtain cellulose in an available form (Non-Patent Documents 2, 3, and 4).

When conversion of cellulose-based biomass into sugars and ethanol is exclusively considered, there are roughly two types of methods for such conversion. One type of method is an acid hydrolysis method whereby cellulose in cellulose-based biomass is hydrolyzed to result in glucose with the use of acids and the like, following which glucose is converted into ethanol by fermentation. Such method has been examined and studied for years. However, reactions are carried out under strongly acidic, high-temperature, and high-pressure conditions, and thus costs of and maintenance costs for apparatuses that can be used under such conditions increase, which has been highly problematic (Non-Patent Documents 2 and 3).

Meanwhile, the other type of method is an enzymatic saccharification method whereby cellulose is degraded into glucose with the use of a cellulose-degrading enzyme (cellulase). Compared with the acid hydrolysis method, the enzymatic saccharification method is advantageous in terms of apparatus structure since reactions can be carried out under moderate conditions. In order to promote degradation, it is necessary for cellulase to come into contact with cellulose contained in a woody biomass. However, the presence of lignin as mentioned above and crystallization of cellulose prevent such contact. Thus, it is necessary to perform some sort of pretreatment prior to an enzymatic reaction. Examples of pretreatment for a method of enzymatically saccharifying a cellulose-based biomass include a variety of methods involving dilute sulfuric acid treatment, alkaline treatment, and fine pulverization. However, no definitive methods have been established (Non-Patent Documents 3 and 5).

A fine pulverization method is a method wherein a biomass is formed into fine particles with a pulverizing device such as a ball mill such that the surface area of cellulose contained in the biomass increases, resulting in ease of cellulose degradation. In such case, it is said that the biomass particle size should be minimized. However, when the biomass particle size is minimized, the energy and cost for such size reduction increase, which is disadvantageous (Non-Patent Document 6). In addition, as an improved fine pulverization method, a method comprising saccharification of a biomass during wet pulverization has been suggested. However, it merely comprises saccharification, and ethanol conversion has not been realized yet (Non-Patent Document 7).

As an example of a fine pulverization method, a method for producing hydrogen from a biomass by subjecting the biomass to mechanical milling in the presence of a transition metal has been developed (Patent Document 1). However, ethanol or the like cannot be produced by this method.
Patent Document 1: JP Patent Publication (Kokai) No. 2006-312690 A
Non-Patent Document 1: Kenji Yamaji (2002), biomass energy characteristics and technology of energy conversion and use, NTS, pp. 3-36
Non-Patent Document 2: Shiro Saka et al. (2001), Biomass/Energy/Environment, IPC, pp. 251-260
Non-Patent Document 3: Jun Sugiura (2002), Biomass energy characteristics and technology of energy conversion and use, NTS, pp. 283-312
Non-Patent Document 4: George P. Philippidis (1996), Handbook on Bioethanol, Taylor & Francis, pp. 253-285
Non-Patent Document 5: The-An Hsu (1996), Handbook on Bioethanol, Taylor & Francis, pp. 183-212
Non-Patent Document 6: Merill A. Millet, et al. (1976), Biotechnol. & Bioeng. Symp., No. 6, pp. 125-153
Non-Patent Document 7: Rick G. Kelsey, et al. (1980), Biotechnology and Bioengineering, No. 22, pp. 1025-1036

### DISCLOSURE OF THE INVENTION

### Problem To Be Solved By the Invention

It is an object of the present invention to develop a conversion method for easily obtaining a useful substance such as ethanol from a cellulose-based biomass at a good yield via enzyme reaction and microbial fermentation.

### Means for Solving Problem

As a result of intensive studies in order to achieve the above object, the present inventors have found that the conversion efficiency can be increased with the use of hard balls or the like when a cellulose-based biomass is converted into sugar with the use of enzyme(s) and then a useful substance such as ethanol with the use of microorganism(s). This has led to the completion of the present invention.

Specifically, the present invention relates to a method for obtaining a product at a high yield by promoting an enzyme reaction and fermentation, comprising introducing a cellulose-based biomass, hard balls, and a reaction solution containing enzyme(s) and microorganism(s) into a single reaction vessel and vibrating the entire reaction vessel so as to allow the balls and the cellulose-based biomass to vigorously come into contact with each other. The present invention is described below in detail.

The present invention relates to the following.

(1) A method for carrying out material conversion, comprising mixing a cellulose-based biomass, hard substance(s), and a reaction solution in a single reaction vessel and carrying out material conversion of a cellulose-based biomass via an enzyme reaction alone or in combination with fermentation while shaking the reaction vessel.

(2) The method for carrying out material conversion according to (1), wherein conversion is carried out with the use of cellulase-based enzyme(s) as an enzyme that converts a cellulose-based biomass and a portion or the entirety of the resultant is further converted into ethanol via fermentation.

(3) The method for carrying out material conversion according to (1) or (2), wherein a cellulose-based biomass is converted into ethanol by carrying out a cellulase-based enzyme reaction and ethanol fermentation in a single reaction vessel.

(4) The method for carrying out material conversion according to any one of (1) to (3), wherein the hard substance(s) is a ball made of zirconia, alumina, stainless steel, iron, fluorine resin, or nylon.

(5) The method for carrying out material conversion according to any one of (1) to (4), wherein the reaction vessel is first vigorously shaken and then shaken in a more moderate manner than before or allowed to stand still once or alternately twice or more in a repetitive manner upon conversion of a cellulose-based biomass.

(6) The method for carrying out material conversion according to any one of (1) to (5), wherein the reaction vessel is provided with a thermal sensor and a jacket capable of circulating warm water around the reaction vessel, and the temperature is controlled by the thermal sensor and the jacket during conversion of a cellulose-based biomass.

(7) The method for carrying out material conversion according to (6), wherein the reaction solution temperature and shaking are interlocked in a manner such that shaking is discontinued when the temperature reaches a predetermined temperature and shaking is resumed when the temperature decreases below the predetermined temperature in a repetitive manner during conversion of a cellulose-based biomass.

(8) The method for carrying out material conversion according to any one of (1) to (7), wherein the reaction vessel is provided with a set containing a pH sensor and a device capable of controlling pH, and the pH of the reaction solution is controlled during conversion of a cellulose-based biomass.

A cellulose-based biomass used for the present conversion method may be in a dry state or wet state and the moisture content thereof is not limited. The size of a biomass is not limited as long as it can be introduced into a vessel used for reaction. However, when it is used in a small size, reaction can be accelerated. In addition, the effects of the present conversion method can be obtained without subjecting a cellulose-based biomass to be used to particular pretreatment. However, a cellulose-based biomass can be subjected to possible pretreatment such as acid treatment, alkaline treatment, fine pulverization, ozone treatment, blasting treatment, or bacterial treatment.

The above cellulose-based biomass is introduced into a reaction vessel. Then, hard balls in an adequate amount, an enzyme solution used for conversion, microorganism(s) used for simultaneous fermentation, and, if necessary, a nutrient source used for fermentation are added thereto. The closed reaction vessel is shaken as vigorously as possible. As a result, an enzyme reaction takes place with better efficiency, due to collision between hard balls, than a reaction in which hard balls are not used. Further, since an enzyme reaction product is immediately consumed by a microorganism coexisting in the vessel, the product is unlikely to cause enzyme reaction inhibition. Consequently, a fermentation product such as ethanol can be obtained in an amount larger than that obtained by general simultaneous fermentation.

Examples of a cellulose-based biomass that can be used include: any herbaceous biomass such as rice straw, rice husk, wheat straw, bagasse, any part of maize, or a different type of plant such as switchgrass; and any woody biomass such as softwood or hardwood chips, wood thinnings, construction debris, or a waste mushroom bed. Further, used paper, cotton, or the like can be used.

As hard balls, balls made of, for example, zirconia, alumina, fluorine resin, or nylon can be preferably used. It is possible to use the same kind of balls or a mixture of balls that are made of different materials and have different sizes. Such balls can be adequately used in accordance with the conditions of a biomass.

As a reaction vessel, a vessel made of any material such as plastic, stainless steel, iron, or a different metal can be used as long as it can be closed to avoid liquid leakage and infiltration of oxygen and it will not be damaged by balls or the like introduced into the same.

When an enzyme reaction and fermentation are carried out, an effective reaction can be performed by providing a thermal sensor, a pH sensor, and the like to a vessel so as to monitor and control the reaction solution temperature and pH. For instance, for temperature control, the temperature can be maintained at a constant level by providing a jacket outside of a reaction vessel for circulation of warm water. In addition, according to the present invention, heat is generated via shaking with hard substance(s). The generated heat can be used as reaction heat. For such purpose, a shaking system that works in conjunction with a thermal sensor is provided to a shaking apparatus. When the temperature reaches a predetermined upper limit temperature as a result of shaking, shaking is discontinued. After the temperature decreases to a predetermined lower limit temperature as a result of discontinuation of shaking, shaking is resumed. Accordingly, waste of energy necessary for heating can be reduced. This is particularly advantageous for production of ethanol, which has been expected to serve as a petroleum substitute.

Regarding a shaking method, shaking can be carried out with the use of any means that allow vigorous shaking to such an extent that a cellulose-based biomass and hard balls are moved in a reaction vessel. When a small reaction vessel is used, a shaking incubator is an adequate means for shaking. Meanwhile, when a large reaction vessel is used, it is effective to use a mixing machine used for mixing in a drum can. Effects of the shaking method can be obtained even in the case of simple reciprocal shaking. However, more favorable effects can be expected in the case of more complex form of shaking at an increased rate.

Enzyme(s) used for conversion may be a marketed product, a culture solution obtained by culturing filamentous fungi, or a purification product of such culture solution, as long as the object of the present invention can be achieved. For instance, in the case of saccharification with cellulase, cellulase and hemicellulase are mixed with a commercially available enzyme or a crude purified enzyme in many cases. The amount of enzyme to be used can be adequately determined. However, it is effective to add 12.5-50 FPU (Filter Paper Unit, filter paper degradation activity) of cellulase containing hemicellulase to a waste mushroom bed. It is also possible to prepare an enzyme solution by suspending an enzyme in water. It is also effective to maintain the pH at 4 to 5 with the use of a buffer containing acetic acid or citric acid. An enzyme solution can be prevented from bacterial contamination by removing bacteria via a filter with a size of 0.45 μm or less. A sugar such as glucose can be obtained from cellulose by terminating the process after cellulase saccharification without microbial fermentation. With the use of hemicellulase such as xylanase, a sugar such as xylose, mannose, arabinose, or galactose from hemicellulose contained in a cellulose-based biomass can be obtained. In addition, with the use of cellulase containing hemicellulase, it is possible to simultaneously obtain a sugar such as cellulose-derived glucose and a sugar such as hemicellulose-derived xylose. The thus obtained sugars can be further converted into a substance such as ethanol and lactic acid via fermentation.

When a sugar is converted into a different substance via fermentation, microorganism(s) is added. For instance, in the case of ethanol fermentation, it is easy and effective to use Saccharomvces cereviciae yeast as a microorganism to be used. However, if a pentose such as hemicellulose-derived xylose is subjected to ethanol fermentation, Pichia stipitis can be used. In addition, salt-tolerant Shizosaccharomyces pombe or the like can be used, depending on conditions. Further, in addition to yeast, any microorganism, including a gene recombinant, such as Zymomonas mobilis capable of causing ethanol fermentation can be used, as long as it can cause ethanol fermentation. When S. cereviciae is used, it can be used in the form of a slant or a cryopreserved product. In addition, a commercially available bakers' yeast may be used. When bakers' yeast is used, good fermentation efficiency is achieved with the use of either dry or raw yeast by directly introducing the yeast into a fermentation system because of the presence of yeast at a high concentration from the beginning of fermentation. When yeast preserved with a slant or the like is used, it is desirable to carry out preculture with the use of a liquid medium prior to simultaneous fermentation so as to increase the amount of yeast and the activity.

In the case of microbial fermentation, fermentation might not take place via shaking with hard balls depending on the microorganism used. In such case, it is possible to solve the problem of lack of fermentation by carrying out vigorous shaking for a certain period of time, followed by moderate shaking or placement under completely still conditions, for promotion of fermentation, or by repeatedly carry out vigorous shaking and moderate shaking or placement under still conditions in an alternate manner.

### Effects of the Invention

According to the present invention, the yield of conversion substance can be significantly increased by carrying out shaking with the addition of hard balls during material conversion with the use of a cellulose-based biomass, and particularly, conversion with enzyme(s) into sugar or conversion with microorganism(s) into ethanol.

This description includes part or all of the contents as disclosed in the description and/or drawings of Japanese Patent Application No. 2007-264041, which is a priority document of the present application.

### BRIEF DESCRIPTION OF THE DRAWINGS

FIG. 1 shows a comparison in terms of the ethanol yield between the present method and the conventional method (without the addition of hard balls).

FIG. 2 shows effects of repetition of placement under still conditions and shaking.

FIG. 3 shows increases in water temperature during shaking.

### BEST MODE FOR CARRYING OUT THE INVENTION

The present invention is hereafter described in greater detail with reference to the following examples, although the technical scope of the present invention is not limited thereto.

### Example 1

### Effects of Ethanol Conversion With the Use of a Maitake Waste Mushroom Bed

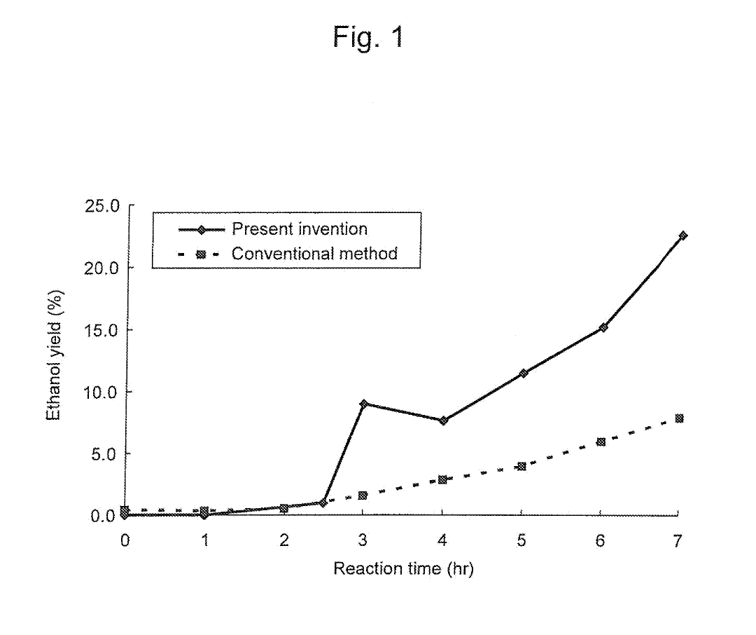

An example of the practice of the present invention with the use of a maitake waste mushroom bed, which is a woody biomass, as a cellulose-based biomass is described below. A maitake waste mushroom bed substantially consists of hardwood sawdust with a moisture content of 60% or more. Such waste mushroom beds (heat-dried) were placed in an amount of 1.0 kg in dry weight in a jacketed stainless steel cylindrical vessel used as a reaction vessel. A cellulase-yeast solution (10 L) was poured thereinto. The composition of the cellulase-yeast solution comprised ion-exchange water containing, as cellulase, GODO-TCD (Godo Shusei Co., Ltd.) (0.6 FPU/ml) and Kameriya yeast (Nisshin Seifun) (1 g/L). Further, zirconia balls 10 mm in diameter (10 kg) were added to the vessel. A control test was conducted without adding zirconia balls. A pH meter, a thermal sensor, and a degassing tube were placed in the vessel. The vessel was closed with a cover and placed in a rocking shaker (RS-100; Seiwa Giken Co., Ltd.). One end of the degassing tube was introduced into water such that the outer air did not enter the vessel. The inner environment of the vessel was adjusted to 37° C. by introducing warm water into the jacket. The shaking rate for the shaker was predetermined at 50 Hz. Then, a shaking operation was initiated. Sampling was adequately performed during conversion reaction. The ethanol concentration in the supernatant was determined by gas chromatography (GB-14; Shimadzu Corporation). FIG. 1 shows the results. The ethanol yield is represented by the percentage of the amount of ethanol obtained with respect to the ideal amount of ethanol calculated based on the amount of cellulose in a waste mushroom bed. As a result, it was found that the ethanol yield obtained by conversion with the present method was 2.9 times greater than that obtained by conversion with a conventional method.

### Example 2

### Effects of Changes in the Shaking Speed During Shaking

Depending on conditions, conversion into ethanol might not take place even if conversion is carried out under conditions similar to the conditions used in Example 1. In order to solve such problem, the shaking speed during conversion was changed. Specifically, shaking was carried out during the first 24 hours of conversion reaction. Thereafter, shaking was terminated and the vessel was further allowed to stand still for 3 to 4 days. Conversion was carried out under conditions such as those in Example 1, except that a raw waste mushroom bed (1.0 kg by dry weight) was used as a substrate and the amount of the cellulase-yeast solution was 5 L. Table 1 shows the results of determination of the ethanol concentration in the supernatant with adequate sampling. As a result, the ethanol yield obtained by conversion with the present method was 1.6 times higher than that obtained by conversion with a conventional method without the addition of hard balls. In addition, an ethanol yield of 0 was used as a precondition in a case in which the shaking speed was not changed.

TABLE 1
Effects of changes in shaking speed on ethanol yield

| | Water | | Ethanol |
|---|---|---|---|
| Test group | volume | Balls | yield (%) |
| Waste mushroom bed + balls (the | 5 L | 20 kg | 60.3 |
| present method) | | | |
| Waste mushroom bed control | 5 L | Not used | 36.6 |
| (conventional method) | | | |

### Example 3

### Effects of Changes in the Shaking Speed During Shaking

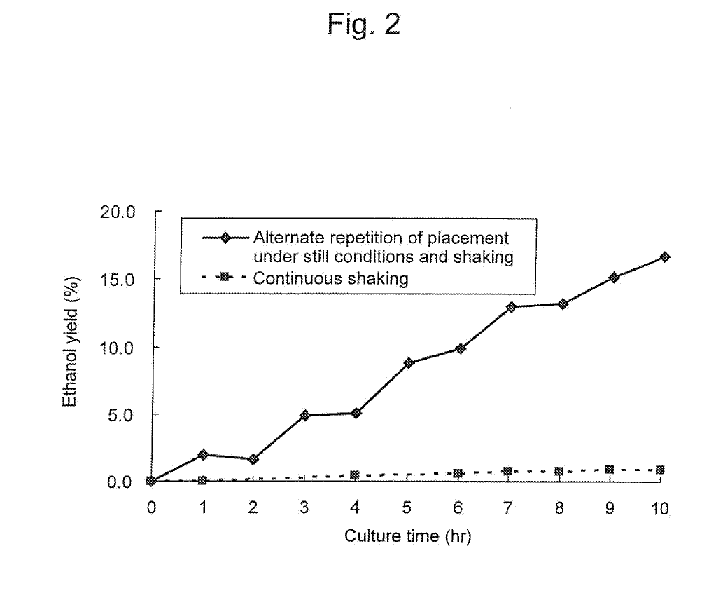

A modified version of the method used in Example 2 was conducted by finely changing the shaking speed. Specifically, shaking was not carried out during the first 1 hour of conversion reaction, and then shaking was carried out for 1 hour and discontinued for 1 hour. Such operation was repeatedly carried out. Conversion was carried out under conditions similar to those used in Examples 1 and 2 except that a dry waste mushroom bed (0.5 kg by dry weight) was used as a substrate and the amount of a cellulase-yeast solution used was 5 L. FIG. 2 shows the results of determination of the ethanol concentration in the supernatant with adequate sampling. Under the above conditions, fermentation was not confirmed as a result of continuous shaking. However, fermentation took place after the vessel had been repeatedly subjected to placement under still conditions and shaking in an alternate manner. Also in the case of continuous shaking, fermentation took place after shaking had been discontinued.

### Example 4

### Use of Heat Generated During Shaking

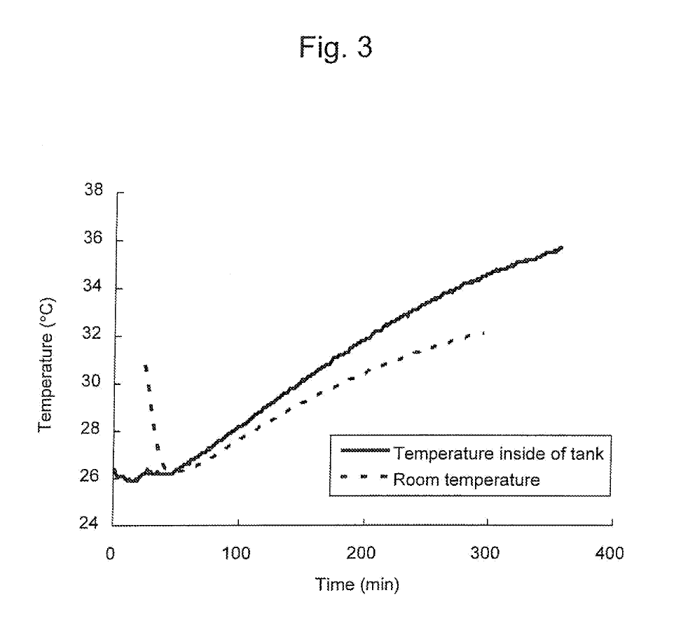

Heat is generated during shaking with hard balls. Therefore, it is effective in terms of cost and energy balance to use heat generated during shaking because it is not necessary to use another heat source. FIG. 3 shows increases in the water temperature of ion-exchange water upon shaking at 50 Hz with the use of the apparatuses used in Examples 1, 2, and 3, provided that ion-exchange water (5 L) and zirconia balls (φ: 10 mm) (20 kg) were placed in a reaction vessel and warm water in the jacket was discarded. It is understood that the temperature increased along with shaking and was always maintained at a level higher than room temperature. In such case, an optimal temperature for simultaneous fermentation of 37° C. can be achieved via shaking alone. Therefore, simultaneous fermentation can be realized even if another heat source is not available.

All publications, patents, and patent applications cited herein are incorporated herein by reference in their entirety.

1. A method for carrying out material conversion, comprising mixing a cellulose-based biomass, hard substance(s), and a reaction solution in a single reaction vessel and carrying out material conversion of a cellulose-based biomass via an enzyme reaction alone or in combination with fermentation while shaking the reaction vessel.

2. The method for carrying out material conversion according to claim 1, wherein conversion is carried out with the use of cellulase-based enzyme(s) as an enzyme that converts a cellulose-based biomass and a portion or the entirety of the resultant is further converted into ethanol via fermentation.

3. The method for carrying out material conversion according to claim 1, wherein a cellulose-based biomass is converted into ethanol by carrying out a cellulase-based enzyme reaction and ethanol fermentation in a single reaction vessel.

4. The method for carrying out material conversion according to claim 1, wherein the hard substance(s) is a ball made of zirconia, alumina, stainless steel, iron, fluorine resin, or nylon.

5. The method for carrying out material conversion according to claim 1, wherein the reaction vessel is first vigorously shaken and then shaken in a more moderate manner than before or allowed to stand still once or alternately twice or more in a repetitive manner upon conversion of a cellulose-based biomass.

6. The method for carrying out material conversion according to claim 1, wherein the reaction vessel is provided with a thermal sensor and a jacket capable of circulating warm water around the reaction vessel, and the temperature is controlled by the thermal sensor and the jacket during conversion of a cellulose-based biomass.

7. The method for carrying out material conversion according to claim 6, wherein the reaction solution temperature and shaking are interlocked in a manner such that shaking is discontinued when the temperature reaches a predetermined temperature and shaking is resumed when the temperature decreases below the predetermined temperature in a repetitive manner during conversion of a cellulose-based biomass.

8. The method for carrying out material conversion according to claim 1, wherein the reaction vessel is provided with a set containing a pH sensor and a device capable of controlling pH, and the pH of the reaction solution is controlled during conversion of a cellulose-based biomass.
